(19) United States
(12) Patent Application Publication
(10) Pub. No.: US 2026/0033814 A1
(43) Pub. Date: Feb. 5, 2026

(54) DEVICE FOR CAPTURING PERSPIRATION FROM A BODY, AND METHOD FOR MANUFACTURING SUCH A DEVICE

(71) Applicant: LINXENS HOLDING, Mantes-la-Jolie (FR)

(72) Inventors: François GERMAIN, Mantes-la-Jolie (FR); Simon VASSAL, Mantes-la-Jolie (FR); Youssef BEN M'BAREK, Mantes-la-Jolie (FR)

(21) Appl. No.: 19/100,042

(22) PCT Filed: Aug. 7, 2023
(86) PCT No.: PCT/FR2023/051252

§ 371 Date: Jan. 30, 2025

(30) Foreign Application Priority Data
Aug. 8, 2022 (FR) 2208157

(51) Int. Cl. A61B 10/00; A61B 5/00; A61B 5/145; A61B 5/1477

(52) U.S. Cl. CPC A61B 10/0064; A61B 5/14517; A61B 5/1477; A61B 5/4266; A61B 5/681; A61B 2562/125

(57) ABSTRACT

The invention relates to a device (100) configured to capture perspiration from a perspiring body and to be attached to an electronic component, comprising a multilayer structure (9) having a contact surface (1) configured to be placed in contact with the body, the structure (9) comprising a microfluidic channel (5) connecting a fluid inlet (3) to a fluid outlet (19), and at least one pair of electrical conductors (27a, 27b), each conductor (27a, 27b) of the pair of conductors (27a, 27b) comprising at a first end, an electrode (23a, 23b) having a surface area extending along a wall of the microfluidic channel (5), and at a second end, a connecting portion (25a, 25b), the connecting portion (25a, 25b) being accessible by an adjacent surface (22a, 22b) or a surface (17) opposite the contact surface (1) and being configured to be electrically coupled to the electronic device.

### FIELD OF THE INVENTION

The present invention relates to a device configured to capture perspiration from a perspiring body and to be attached to an electronic device, as well as a method for manufacturing said device.

### TECHNOLOGICAL BACKGROUND

Devices are known in the art having the aim of providing measurements relating to the perspiration of a perspiring body. Perspiration is a biological thermoregulation mechanism prevalent in mammals, during which a liquid is secreted by glands and expelled through the pores of the skin. The parameters of this biological phenomenon can provide useful medical indications about the condition, the health and the environment of the perspiring body in question.

Thus, it can be desirable to obtain information on the volumetric flow rate of the perspiration of a body, on the temperature of the perspiration liquid, or on the composition of the perspiration liquid, for example the concentration of urea, lactate or minerals, such as sodium chloride (NaCl). This information can be useful for medical purposes, for example by evaluating the data during long-duration monitoring, as well as in real time, for example in the scope of physical exercise and/or when the body is subjected to high temperatures.

In particular, during a physical exercise session, real-time monitoring of the perspiration parameters of the perspiring body can offer more accurate information on the state of the body and make it possible to adapt the effort or the environment. For example, monitoring perspiration parameters can serve to obtain optimised hydration instructions to achieve a desired physical performance.

Thus, to capture the perspiration of a perspiring body, various appliances have been proposed, aiming to obtain one of the measurements indicated above. These devices generally comprise at least one capture device and at least one capture measurement and analysis device. The capture device comprises a contact surface configured to be placed in contact with the body, so as to be able to perform a capture operation on a parameter of the liquid secreted by one or more pores of the skin of the perspiring body. The measurement and analysis device is interfaced with the capture device, so as to measure the capture and to process and analyse the measurement, in order to subsequently communicate it, for example, to a user.

### TECHNICAL PROBLEM

In the scope of physical exercise, the device for capturing perspiration is subject to a high risk of physical degradation. For example, the device can be confronted with a significant quantity of sudden movements and shaking, and can also suffer from vibrations, shocks, or exposure to precipitation or be contaminated by the natural environment (dust, mud, etc.). Thus, the device must both have a good level of resistance to external effects, and have a sufficient level of accuracy, and be comfortable to use.

### AIM OF THE INVENTION

Given the above, an aim of the invention is to provide a device for capturing perspiration, which is, at the same time, reliable, compact and economical to manufacture. In particular, the invention aims to obtain a device for capturing perspiration which is consumable, i.e. replaceable at a low cost from a physical exercise session.

This aim is achieved with a device configured to capture perspiration from a perspiring body and to be attached to an electronic device. The device comprises a multilayer structure having a contact surface configured to be placed in contact with the body, the structure comprising an inner microfluidic channel extending through the structure and fluidly connecting at least one fluid inlet to at least one fluid outlet. The inlet is located at said contact surface and is configured to interface with a surface of the body comprising at least one pore, and the outlet is located at another surface different from said contact surface.

The structure moreover comprises at least one pair of electrical conductors, in particular copper conductors, each conductor of the pair of conductors comprising, at a first end, an electrode having a surface area(S) extending along a wall of the microfluidic channel, and at a second end, a connecting portion, the connecting portion being accessible by an adjacent surface or a surface opposite the contact surface and being configured to be electrically coupled to the electronic device. For example, one or more openings can be configured in the adjacent surface or a surface opposite the contact surface to receive connecting elements from the electronic device.

The implementation of such a device with an inlet of the microfluidic channel enables the introduction of sweat in liquid form into the microfluidic channel, which guides, by capillary force, a laminar flow of the liquid to the outlet, along the electrodes. The inclusion of such a microfluidic channel, as well as electrical conductors in a multilayer structure, enables a manufacturing of the device by microfabrication techniques which benefit from significant economies of scale, for example reel-to-reel methods. Thus, the device can be manufactured sufficiently economically to be used consumably during a physical exercise session.

In addition, an arrangement of connecting portions of the conductors, so as to be accessible by a surface adjacent to or opposite the contact surface of the multilayer structure enables the attachment to an electronic device configured to measure a resistance and/or a conductance between the electrodes, without encumbering the contact surface. Thus, the simple operation and comfort of the device are preserved.

In an embodiment, the microfluidic channel can extend in a straight line from the fluid inlet to the fluid outlet of the structure.

Thus, the amount of sweat entering the microfluidic channel accesses the outlet through a direct path, and the liquid can more rapidly come into contact with the pair(s) of electrodes, and be ejected more rapidly through the outlet. The risk of clogging can thus be reduced, and the device, in particular the guiding of the liquid through the microfluidic channel, can be made more reliable.

In an embodiment, the contact surface can comprise at least one groove for directing perspiration to the fluid inlet.

The groove can be used to guide the sweat flowing through additional pores to the fluid inlet of the device. Thus, the quantity of liquid received by the device can be greater and the information obtained more complete.

In an embodiment, the respective electrodes of the pair of conductors can extend along one same wall of the microfluidic channel.

This configuration is simple to produce by a microfabrication method, for example, a reel-to-reel-type method, as the electrical conductors can be arranged in the same plane. Thus, this configuration can only require the implementation of a layer of conductive material, for example, copper.

In an alternative embodiment, the respective electrodes of the pair of conductors can extend along opposite walls of the microfluidic channel.

In this configuration, two walls of the channel can be provided with electrodes. The electrodes can be opposite one another and thus implement a lesser quantity of sweat, in particular compared with a configuration of electrodes juxtaposed in one same plane. This can enable a more accurate measurement.

In an embodiment, the multilayer structure can have a parallelepiped shape, in particular, a flat, rectangular parallelepiped shape.

A parallelepiped shape can enable a simple and robust fixing at four corners. A flat shape can moreover guarantee the comfort of the user when the device is in contact with the skin.

In an embodiment, the ratio between the distance (L) separating the electrodes and the surface area(S) of the electrodes can be between 0.01 and 0.1 m\hat{ }-1, in particular between 0.03 and 0.05 m\hat{ }-1.

According to a discovery of the inventors, in this range, the ratio between the conductivity of sweat and its molar concentration of sodium chloride is linear. Thus, a particularly simple and accurate measurement of the rate of sodium chloride in sweat can be obtained.

In an embodiment, the multilayer structure of the device can comprise a base layer, an intermediate layer and a cover layer. The base layer can comprise the contact surface configured to be placed in contact with the body. The intermediate layer can be disposed between the base layer and the cover layer.

In an embodiment, the microfluidic channel can be provided in the intermediate layer. The intermediate layer can be deposited directly on the surface of the base layer, which is opposite said contact surface of the base layer.

In an embodiment, at least one electrode, in particular the respective electrodes of the pair of conductors, can be directly deposited on the surface of the base layer, which is opposite said contact surface of the base layer. This arrangement of electrodes, which are deposited on the base layer in the microfluidic channel, makes it possible to provide a more compact device, as it makes it possible to reduce the number of layers necessary to form the device. Furthermore, this arrangement makes it possible to reduce manufacturing costs, as the deposition of the electrodes and conductors on the base layer can be obtained with microfabrication techniques of the “reel-to-reel processing” type. The reduction in the manufacturing costs of the multilayer structure is particularly advantageous for manufacturing a consumable device, that the user can replace after each use, in particular for hygiene reasons.

In an embodiment, in a cross-section of the device along the thickness of the multilayer structure, the microfluidic channel can extend in the intermediate layer from the base layer to the cover layer. The microfluidic channel can thus have a height equal to the thickness of the intermediate layer.

In an embodiment, the device can be characterised in that the multilayer structure comprises only three layers. The device is thus simple to manufacture, which reduces its cost. Furthermore, comprising only three layers, a compact device is obtained, which is particularly advantageous for a portable device. A less cumbersome device can thus be made available to the user.

In an embodiment, the device can be characterised by the absence of electronic components. The device can thus be manufactured at a lower cost, which is all the more advantageous for producing a single-use device or consumable device.

The aim of the invention is moreover achieved, thanks to a perspiration measurement appliance, comprising a microfluidic device according to one of the embodiments described above and an electronic device, the microfluidic device being attached to the electronic device, the connecting portions of at least one pair of conductors of the microfluidic device being electrically coupled to the electronic device, and the electronic device being configured to measure an electrical conductance and/or an electrical resistance between the electrodes of the device.

This perspiration measurement appliance has the advantage of having separated the electronic elements for the electrical measurement and signal transformation of the capture device in direct contact with the skin of the user.

In an embodiment of the appliance, the appliance can comprise a wristwatch attached to the electronic device and configured to be worn around a wrist of a user. In particular, the wristband can be configured, such that a contact surface of the device configured to capture perspiration is arranged along a surface of the skin of the user.

Such an installation of the measurement appliance can avoid the discomfort of movements during a physical exercise session, as well as enable an immediate visual access to the appliance and a comfortable use in the way of a watch.

In an embodiment, the electronic device can comprise a transmission device configured to transmit the measurements and/or receive instructions.

Thus, the measurements of the electronic device can be received, processed, analysed and recorded by a third-party appliance not exposed to external effects in the immediate environment of the user during a physical exercise session.

The aim of the invention is moreover achieved, thanks to a method for manufacturing a device configured to capture the perspiration of a perspiring body according to one of the embodiments described above.

The method comprises the steps of: providing a base layer and manufacturing at least one pair of electrical conductors on a base layer, each conductor comprising at a first end, an electrode, and at a second end, a connecting portion; providing an additional layer comprising at least one cut region forming a microfluidic channel; and assembling, in particular by colamination, the base layer and said at least one additional layer such that the electrodes are arranged in the cut region.

This method makes it possible to economically and rapidly obtain a device having the features and advantages described above.

In an embodiment, the method can comprise an additional step of closing the microfluidic channel with another additional layer, in particular by colamination.

In an embodiment, the method can comprise an additional step of producing a through hole in the base layer forming an inlet to the microfluidic channel.

In an embodiment of the method, the cut region forming a microfluidic channel can comprise an access point to the through hole forming an inlet.

In an embodiment, step a) of the method according to the invention can moreover comprise the steps of
i) Providing a base layer
ii) Covering the base layer with a copper layer
iii) Applying a photosensitive resin layer at least on the copper layer
iv) Selectively exposing the photosensitive resin to a light beam, in particular an ultraviolet beam, so as to delimit a path of the conductors
v) Dissolving the unexposed photosensitive resin, and
vi) Chemically etching the copper layer, so as to obtain a base-conductors complex.

This method can be implemented on a large scale, and thus lead to sufficient cost efficiency to propose a device for capturing consumable perspiration.

In another embodiment, step a) of the method according to the invention can comprise the steps of
i) Providing a base layer, and
ii) Printing the conductors on the base layer.

In an embodiment, the method can moreover comprise an additional step of
x) Plating of conductors, in particular electrodes, with at least one metal, preferably a metal selected from nickel, gold, silver, and palladium, or with a metal alloy, preferably a metal alloy comprising at least one metal selected from among nickel, gold, silver, and palladium.

Such a plating, also called metallisation, makes it possible to obtain electrodes and/or connecting portions with a lower contact resistance, and a higher corrosion resistance. Thus, a method implementing this step makes it possible to result in a device for capturing perspiration offering a more reliable and more accurate measurement.

In an embodiment, the plating of step x) can be a plating with a nickel layer covered with a gold layer. The properties of such a plating are particularly advantageous.

In an embodiment, step x) can take place after step vi).

### BRIEF DESCRIPTION OF THE DRAWINGS

The aims, features and advantages of the invention such as exposed above will be more exhaustively understood and assessed by studying the following, more detailed description relating to the invention, as well as the accompanying drawings.

FIG. 1 illustrates a perspiration measurement appliance according to an embodiment of the invention, comprising a device for capturing perspiration.

FIG. 2A is a perspective view of the device of FIG. 1, partially open at the microfluidic channel.

FIG. 2B is a perspective view of the device of FIG. 1, highlighting the electrical conductors.

FIG. 3A represents cross-sections of the device of FIG. 1 along the section axis A-A of FIG. 2A.

FIG. 3B represents cross-sections of the device of FIG. 1 along the section axis B-B of FIG. 2A.

FIG. 3C represents cross-sections of the device of FIG. 1 along the section axis C-C of FIG. 2A.

FIG. 4A represents cross-sections of a device for capturing perspiration according to a second embodiment of the invention.

FIG. 4B represents cross-sections of a device for capturing perspiration according to a second embodiment of the invention.

FIG. 5 schematically represents the successive steps of a method for manufacturing a device according to an embodiment of the invention.

In order to make the figures legible, the elements illustrated are not necessarily represented to scale, not relative to one another, nor in their relative Cartesian dimensions.

### DETAILED DESCRIPTION OF THE DRAWINGS

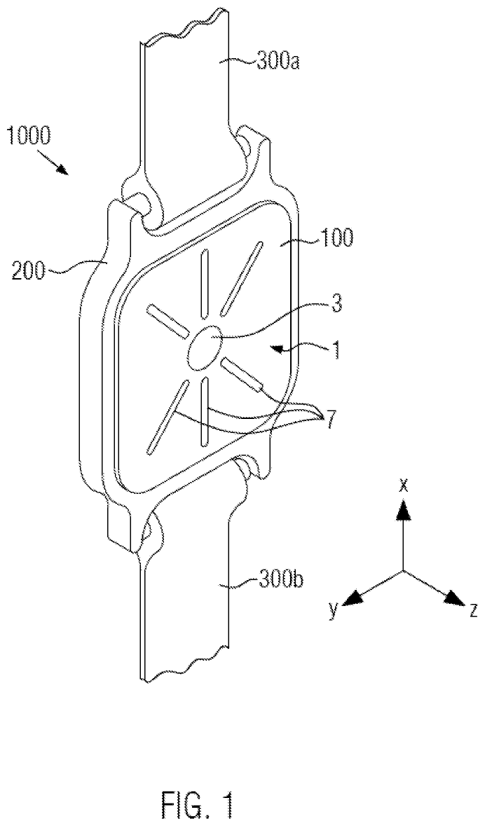

FIG. 1 illustrates a perspiration measurement device according to an embodiment of the invention; The appliance 1000 comprises a device for capturing perspiration 100 and an electronic device 200. The device 100, in this embodiment, has a flat rectangular parallelepiped shape, having length and width dimensions in two Cartesian directions x,y which are of the same order of magnitude, and a height dimension in a third Cartesian direction z which is much less, for example, at least 5 times less than the length and/or width dimensions.

In other embodiments, it can also be considered to provide a device for capturing perspiration 100 with a cross-section in the x-y plane which is triangular, hexagonal, octagonal, and/or with rounded or chamfered corners.

In the embodiment of FIG. 1, the electronic device 200 also has substantially a shape of a flat rectangular parallelepiped, having length and width dimensions in two Cartesian directions x,y that are of the same order of magnitude and a height dimension in a third Cartesian direction z which is much less, for example, at least 5 times less than the length and/or width dimensions.

The dimensions of the electronic device 200 are, one by one, greater than those of the device, in particular greater by 10 to 30%, such that the device 100 can be received in an opening of a space excavated in the electronic device 200, such as illustrated in FIG. 1.

In the view of FIG. 1, the device 100 is received in a space excavated in the electronic device 200 and is attached thereto by an attachment means by form-fitting not represented in FIG. 1, for example a snap-fitting means or a bonding. In this position, the device 100 is electrically coupled to the electronic device 200.

The appliance 1000 moreover comprises two parts 300a, 300b of a wristwatch, attached respectively at two opposed thin portions of the electronic device 200. Thus, the appliance 1000 can be comfortably worn on a limb of the user, for example, on a wrist or an ankle. The two parts 300a, 300b of the wristwatch are arranged, such that the contact surface 1 of the device 100, received in an excavated space, is arranged along a surface of the skin of the user. I.e. the device 100 is face-down with respect to the field of vision of the user.

The electronic device 200 comprises a transmission device (not represented) for transmitting the measurements relating to the electrical coupling and/or for receiving instructions. The envelope of the electronic device 200 is made of a rigid and lightweight material.

The device 100 has a contact surface 1 configured to be placed in contact with a perspiring body. The surface 1 is flat and, when the device 100 is attached to the electronic device 200, emerges slightly in a direction z from the electronic device 200. I.e. the device 100 exceeds slightly, in particular by 1% to 10% of the height of the device 200, from the excavated space of the device 200. Thus, the device 100 is brought into contact with the skin of the perspiring body of a user when the appliance 1000 is worn by the user.

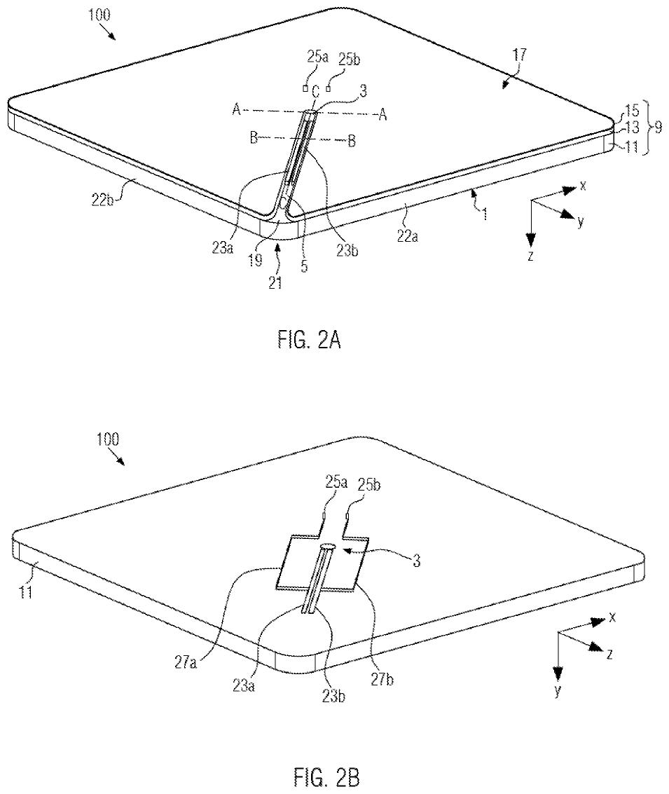

The surface 1 moreover has a fluid inlet 3 with a microfluidic channel 5 (which can be seen in FIG. 2A), the inlet 3 being located at the centre of the surface 1. The contact surface 1 also has a pattern of a plurality of grooves 7. The pattern is centred on the inlet 3, each groove 7 of the pattern being connected to the inlet 3. The groove 7 pattern makes it possible to more effectively direct a high quantity of sweat to the fluid inlet 3.

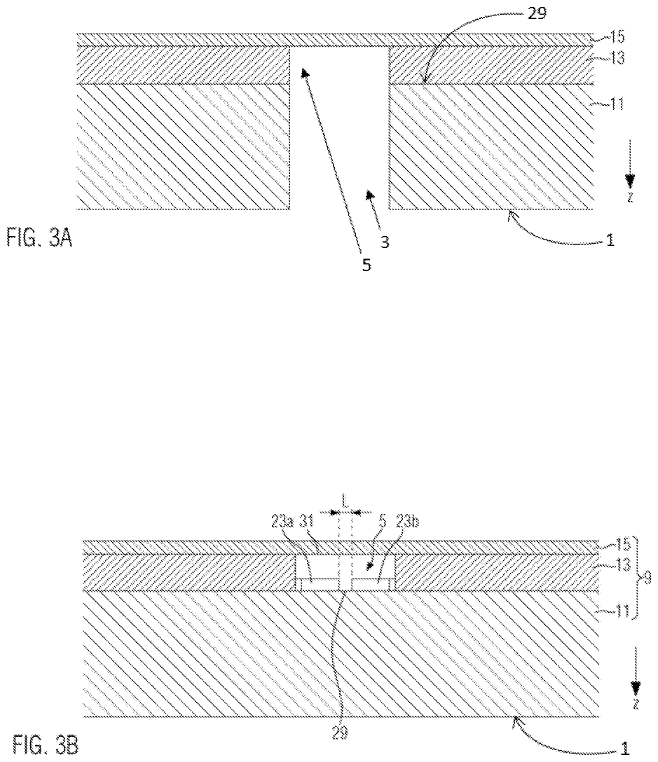

The structure and the operation of the device 100 of FIG. 1 are described in more detail below, in reference to FIGS. 2A, 2B and 3A-3C. In particular, it will be described that the device 100 does not comprise electronic components, for example chips. Thus, in the measurement appliance 1000, the electronic elements for electrical measurement and signal transformation are offset from the capture device 100, in direct contact with the skin of the user, towards the electronic device 200. Thanks to the transmission device included in the electronic device 200, the processing, analysis and storage of the measured data can be performed by a third-party appliance not exposed to the external effects of the immediate environment of the user during a physical exercise session. At the same time, the appliance 1000 comprising the electronic device 200 is flexible and comfortable to wear, as well as simple to manufacture.

FIG. 2A is a perspective view of the device 100 for capturing perspiration. For purposes of illustration and understanding, the view of FIG. 2A is partially cut along the microfluidic channel 5, in order to identify the path of the microfluidic channel 5 inside the device 100.

The device 100 has a multilayer structure 9, comprising a base layer 11, an intermediate layer 13, and a cover layer 15. Preferably, the layers 11, 13, 15 are layers of a thin material, for example chosen from among a polyethylene terephthalate (PET), a glass-epoxy composite film, a polyimide (PI), a polyethylene (PE), a polyethylene naphthalate (PEN), an adhesive or a paper. The layers 11, 13, 15 can be layers of materials identical to or different from one other. In particular, in an embodiment, the layer 13 can be a layer of an adhesive material, for example a pressure-sensitive adhesive, PSA, to hold the layers 11 and 15 together. The base layer 11 has, on the hidden face which cannot be seen in FIG. 2A, the contact surface 1 and comprises the inlet 3 to the microfluidic channel 5. The cover layer 15 has an external connecting surface 17. Each of the layers has a thickness of between 20 μm and 200 μm, and preferably, the base layer 11 is thicker than the intermediate layer 13, and the layer 13 is thicker than the cover layer 15. For example, the base layer 11 can have a thickness of 120 μm, the intermediate layer 13 a thickness of 50 μm and the cover layer 15 a thickness of 20 μm.

The multilayer structure 9 comprises the microfluidic channel 5 extending inside the structure 9 in an x-y plane. In particular, the channel 5 is located at the intermediate layer 13, between the base layer 11 and the cover layer 15, and extends linearly, i.e. in a straight line, from the inlet 3 to the centre of the parallelepiped of the device 100 to the fluid outlet 19 of the channel. The channel 5 can have a thickness of 20 μm to 200 μm and a width in the x-y plane of 200 μm to 5 mm. The outlet 19 is arranged at a corner 21a of the parallelepiped of the device 100, at the intersection of two surfaces 22a, 22b adjacent to the contact surface 1. The channel 5 has a lower wall corresponding to a portion of the surface 29 (which can be seen in FIGS. 3B and 3C) of the base layer 11 opposite the contact surface 1, a top wall corresponding to a portion of the surface 31 (which can be seen in FIGS. 3B and 3C) of the cover layer 15 opposite the external connecting surface 17, and two opposite side walls internal to the intermediate layer 13.

FIG. 2A also shows two electrodes 23a, 23b, disposed in the microfluidic channel 5, so as to extend along the side walls of said channel 5. Moreover, two connecting portions 25a, 25b are arranged in the structure 9, so as to be accessible by the external connecting surface 17. For example, one or more openings can be configured in the external connecting surface 17 to receive connecting elements from the electronic device 200. The electrodes 23a, 23b correspond to the respective first ends and the portions 25a, 25b correspond to the respective second ends of two electrical conductors 27a, 27b, which will be described in more detail in reference to FIG. 2B.

The external connecting surface 17 and the contact surface 1 correspond to opposite operational surfaces of the parallelepiped of the device 100. While the contact surface 1 is configured to be brought into contact with the skin of a perspiring body, the external connecting surface 17 is engaged with the electronic device 200 (see FIG. 1), so as to contact the connecting portions 25a, 25b with the electronics of the electronic device 200 and establish an electrical coupling.

The device 100, according to this embodiment, comprises a microfluidic channel 5 connecting an inlet 3 to an outlet 19. Alternative embodiments with several inlets leading to the channel 5, and/or several outlets for the escape of the liquid from the channel 5, can be considered without departing from the spirit of the invention. Likewise, it can be considered to arrange several microfluidic channels in the device 100, for example four channels respectively connecting four inlets and extending respectively towards the four corners of the parallelepiped of the device 100, each channel being provided with a pair of electrodes, as well as connecting portions.

FIG. 2B shows a view of a base-conductors complex of the device 100, i.e. without the intermediate layer 13 and without the cover layer 15, thus highlighting the path of the conductors 27a, 27b. The electrical conductors 27a, 27b are disposed on the surface 29 (which can be seen in FIGS. 3B and 3C) opposite the contact surface 1 of the base layer 11 of the multilayer structure 9. The conductors 27a, 27b are made of an electrically conductive material, preferably copper (Cu) and comprise, at their respective first ends, the electrodes 23a, 23b and at their respective second ends, connecting portions 25a, 25b. In particular, the conductors 27a, 27b are made from the same copper layer formed on the base layer 11, the copper having been chemically etched to obtain the desired shape.

The connecting portions 25a, 25b are exposed parts of the conductors 27a, 27b. For example, the layers 13 and 15 are open at the connecting portions 25a, 25b in order to enable a physical connection with the electronic device 200 (see FIG. 1) through the external surface 17. Thus, an electrical contact with the electronic device 200 can be established. The electrodes 23a, 23b are expanded portions of the conductors 27a, 27b, disposed along walls of the microfluidic channel 5.

In order to improve their functionality, the electrodes 23a, 23b and the connecting portions 25a, 25b of the conductors 27a, 27b are plated, or metallised, with at least one metal or with a metal alloy, the metals preferably being selected from among nickel, gold, silver, and palladium. In a preferred embodiment, the electrodes 23a, 23b are plated with a 10 nm to 10 μm nickel layer, which in turn is covered with a 10 nm to 10 μm gold layer. In alternative embodiments, the electrodes 23a, 23b are plated with a layer of nickel covered with a layer of silver, or a layer of nickel covered with a layer of palladium, or a layer of nickel covered with a layer of gold, itself covered with a layer of palladium. Thus, the contact resistance can be reduced and/or the corrosion resistance increased.

The arrangement of connecting portions 25a, 25b at one end of the conductors 27a, 27b, so as to be accessible by the external surface 17 of the multilayer structure 9, enables the simple attachment to the electronic device 200 (see FIG. 1) to measure a resistance and/or conductance between the electrodes, without encumbering the contact surface 1. Thus, the operation and the comfort of the device are preserved.

FIG. 2B also shows that the fluid inlet 3 at the centre of the parallelepiped of the device 100 corresponds, in this embodiment, to a cylindrical perforation through the thickness, i.e. through the height, in a direction z, of the base layer 11.

Thus, a quantity of sweat received by the fluid inlet 3 is brought into the microfluidic channel 5 and guided by capillary force along the channel 5, then escapes through the outlet 19. When sweat passes between the electrodes 23a, 23b, a resistance and/or conductance between the terminals of the electrodes can be measured. From the resistance R or the conductance G, and from the ratio between the surface area S of the electrodes 23a, 23b and the distance L separating said electrodes 23a, 23b, called S/L ratio, the electrical conductivity o of the sweat is calculated. From the conductivity o of the liquid, the molar concentration C_NaCl of sodium chloride can be empirically approximated.

The S/L ratio of the electrodes 23a, 23b disposed in the channel is predetermined and known. In this embodiment, the S/L ratio is between 0.01 m\hat{ }-1 and 0.1 m\hat{ }-1, in particular between 0.03 m\hat{ }-1 and 0.05 m\hat{ }-1. Preferably, the ratio approaches 4/100, i.e. 0.04 m\hat{ }-1. In the scope of in-depth investigations by the inventors, it has been determined that the relationship between conductivity o and sodium chloride concentration C_NaCl is advantageously linear and determining in this range.

FIGS. 3A, 3B and 3C represent cross-sections of the device 100 along, respectively, the respective section axes A-A, B-B and C-C, such as represented in FIG. 2A.

The three cross-sections are centred at the microfluidic channel 5 along, respectively, the axis A of FIG. 2A, and the x-z plane. The cross-section 3A along the axis A-A is centred on the inlet 3 to the microfluidic channel 5. The inlet 3 is a central perforation through the base layer 11. While the perforation of the base layer 11 also passes through the intermediate layer 13, the cover layer 15 is not perforated.

FIGS. 3B and 3C moreover represent the two electrodes 23a, 23b disposed on one same surface 29. The surface 29 is opposite the contact surface 1 of the base layer 11 of the multilayer structure 9. The electrodes 23a, 23b are disposed so as to be separated by a distance L. Thus, the electrodes 23a, 23b are arranged in the same x-z plane, at the intermediate layer 13. The cover layer 15 covers the intermediate layer 13 and provides an additional wall to the microfluidic channel 5, closing the channel 5. Thus, this embodiment is economical, as it is simple to produce by a microfabrication method.

In an alternative embodiment, it can be considered to dispose the two electrodes 23a, 23b on the cover layer 15, on one same surface of the microfluidic channel 5 wall, for example on the surface 31 opposite the external connecting surface 17.

The structure of the device 100 described above enables a manufacture by microfabrication techniques, in particular by reel-to-reel methods, and therefore benefit from significant economies of scale. Thus, the device can be manufactured sufficiently economically to be used consumably during a physical exercise session.

Moreover, the arrangement of connecting portions 25a, 25b at one end of the conductors 27a, 27b, so as to be accessible by the surface 17 opposite the contact surface 1 of the multilayer structure 9 enables the attachment to the electronic device 200 without encumbering the contact surface. Thus, the simple operation and the comfort of the device are preserved.

In the embodiment according to FIGS. 1 to 3C, the device 100 is provided with a pair of electrical conductors 27a, 27b having respective electrodes 23a, 23b. In alternative embodiments, it can be considered to arrange several pairs of electrodes in sequence along one same microfluidic channel, for example three pairs. By arranging several pairs of electrodes in a row, a measurement time differential can be established when a quantity of sweat passes successively through the pairs of electrodes. The time differential can, for example, be used to determine, approximate, or divert a liquid flow velocity and/or a liquid volume flow rate.

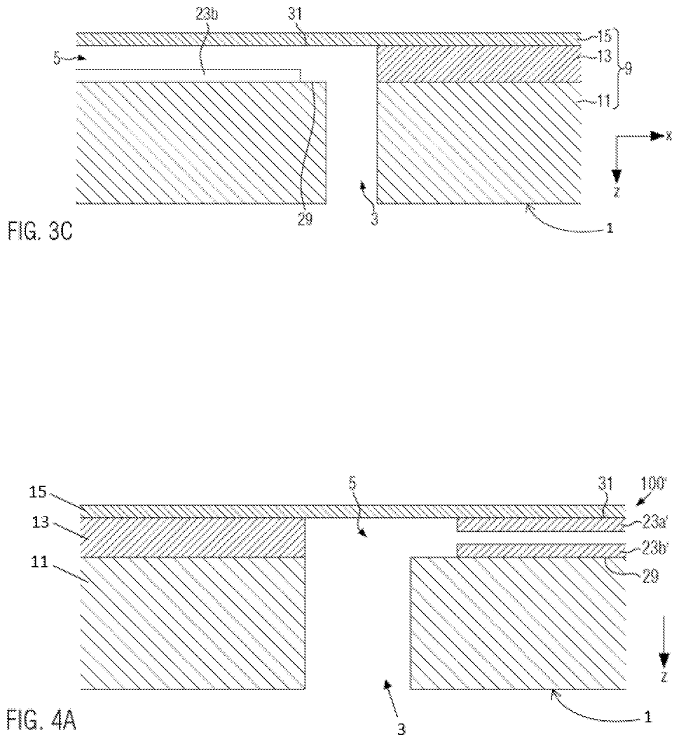

FIGS. 4A and 4B illustrate a device 100′ of another embodiment of the invention. Only features which diverge with respect to the device 100 are described, the features being able to be considered as equivalent to those described above relative to the device 100.

The device 100′ also comprises two electrodes 23a′ and 23b′. Instead of being disposed on one same wall of the microfluidic channel 5′ corresponding to the channel 5 of the device 100, they are disposed on two opposite walls. In particular, they are disposed on the opposite surfaces 29 and 31 of the device 100. Alternatively, it can also be considered to dispose the electrodes on two side walls of the channel 5′. Thus, the microquantities of sweat enclosed between the two electrodes 23a′, 23b′ are smaller than, for example, between the electrodes 23a, 23b. This enable a more accurate measurement.

An embodiment of a method for manufacturing, according to the invention, a device configured to capture perspiration from a perspiring body, and to be attached to an electronic device, for example the device 100, is described below. The method comprises a first step of manufacturing at least one pair of electrical conductors on a base layer, each conductor comprising at a first end, an electrode, and at a second end, a connecting portion.

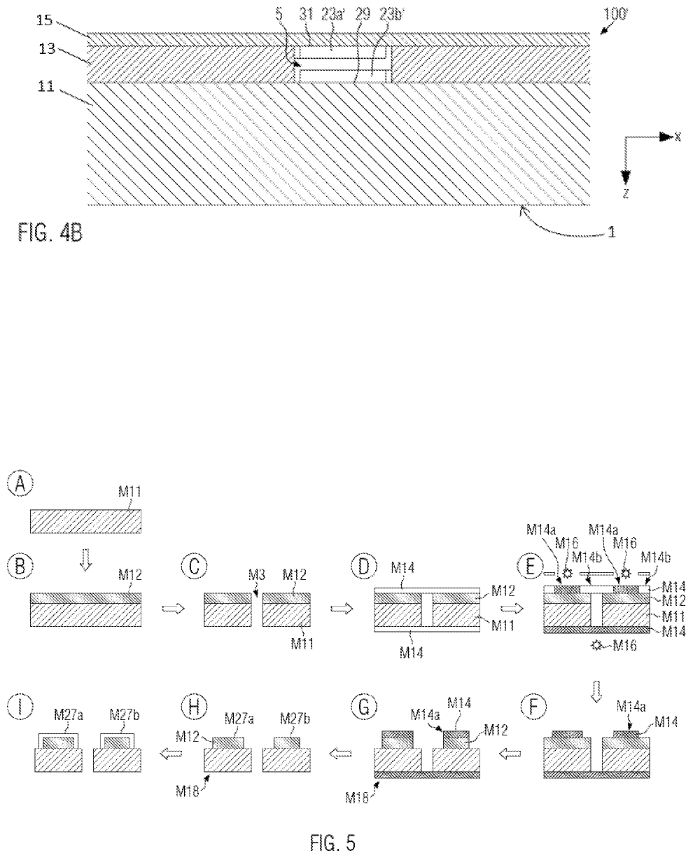

FIG. 5 schematically represents the successive steps of a method for carrying out the first step by photolithography.

In particular, the method starts with a step A of providing a base layer M11. For example, the base layer M11 can be the base layer 11. Preferably, the layer M11 is a layer of a material chosen from among a polyethylene terephthalate (PET), a glass-epoxy composite film, a polyimide (PI), a polyethylene (PE), a polyethylene naphthalate (PEN), or a paper. The layer M11 is preferably thin, in particular of a thickness of between 20 μm and 200 μm.

The base layer M11 is, in a step B, covered with a layer of copper (Cu) M12 of a thickness of between 1 μm and 100 μm, preferably between 12 μm and 70 μm. Alternatively, it is possible to use a “copperclad”-type material, in which the copper layer M12 is attached to the base layer M11 by vacuum deposition, by hot assembly, or by assembly with adhesive. In a step C, a through hole M3 is made in the base layer M11. In this embodiment, the base layer M11 covered with the copper layer M12 is mechanically perforated at a desired location to form the hole M3 forming an inlet to a microfluidic channel. For example, the hole M3 can form the fluid inlet 3 perforated through the copper-coated base layer 11.

Then, in a step D, a layer of photosensitive resin M14 is applied to the base layer M11 coated with copper M12 at least on the copper side, alternately on both sides.

In a step E, the photosensitive resin M14 is selectively polymerised. In this step, called insolation step, portions of the applied photosensitive resin are exposed to a light beam M16, in particular an ultraviolet beam, delimiting on the copper side of the base layer M11, polymerised zones M14a and non-polymerised zones M14b. The polymerised zones M14a represent the desired paths of electrical conductors formed from the copper layer M12 on the base layer M11. For example, the conductors 27a, 27b are delimited on the layer 11 such as illustrated in FIG. 2B. The resin M14 located above the parts of the copper layer M12 not necessary for the formation of electrical conductors is not insolated, i.e. is not exposed to the light beam, and therefore non-polymerised M14b.

In a step F, called development step, the part of the photosensitive resin layer M14 which is non-insolated, and therefore non-polymerised and non-crosslinked, is dissolved, such that the polymerised zones M14a of the resin M14 remain.

In a step G, the copper layer M12 is chemically etched. In this step, the polymerised M14a photosensitive resin M14a remaining on the copper M12 selectively protects portions of the copper from etching. Thus, only the copper non-protected by the photosensitive resin M12 is etched. An electrical conductor circuit part is thus obtained on the base layer. Thus, a base-conductors complex M18 is formed, for example the base-conductors complex illustrated in FIG. 2B.

In a step H, a chemical stripping of the base-conductors complex M18, is carried out in order to remove the remaining exposed, i.e. polymerised, portions of photosensitive resin from the base layer M11 and the copper M12 forming the conductor circuit. This makes it possible, in particular, to free up the surface of the copper layer M12 and obtain the conductors M27a, M27b. For example, the conductors 27a, 27b are obtained on the base layer 11.

In a step I, carried out after the etching and stripping steps, the electrical conductors M27a, M27b are plated with at least one metal with or a metal alloy, the metals being, for example, selected from among nickel, gold, silver, and palladium. Preferably, the electrical conductors M27a, M27b are plated with a layer of nickel covered with a layer of gold. Optionally, only certain portions of the conductors, for example the electrodes 23a, 23b and the connecting portions 25a, 25b, are plated.

In another embodiment of the manufacturing method, the first step of the method is not carried out by photolithography, but by an additive printing method. Printing can be carried out by conductive ink screen printing. Alternatively, printing can be carried out by inkjet. In this embodiment, the electrical conductors are printed on a provided base layer. Each conductor is printed on the base layer, so as to comprise at least one electrode and at least one connecting portion.

For example, the conductors 27a, 27b are printed on the base layer 11. Alternatively, a conductor 27a is printed on the base layer 11 and a conductor 27b is printed on another base layer, for example the cover layer 15. The two layers thus printed can thus be assembled, for example by an adhesive intermediate layer, such as the intermediate layer 13. By cutting the intermediate layer as described below, the device 100′ of FIGS. 4A, 4B can be obtained.

In another example, the conductors 27a, 27b are both etched on the base layer 11. Alternatively, a conductor 27a is etched on the base layer 11 and a conductor 27b is etched on another base layer, for example the cover layer 15. The two layers thus manufactured can thus be assembled, for example by an adhesive intermediate layer, such as the intermediate layer 13. By cutting the intermediate layer as described below, the device 100′ of FIGS. 4A, 4B can be obtained.

In a second step of the method not represented in FIG. 5, an additional layer comprising at least one cut region forming a microfluidic channel is provided. The at least one additional layer is cut, so as to delimit a microfluidic channel, to form the at least one microfluidic channel of the device.

Optionally, the at least one additional layer can also be cut, so as to delimit an access point at a second end of the electrical conductors. Moreover, the cut region forming the microfluidic channel comprises an access point to the through hole forming the inlet of step C.

For example, the intermediate layer 13 can be cut to form the microfluidic channel 5 and to cover the base layer 11, as well as to comprise an access point to the fluid inlet 3. In particular, the microfluidic channel delimited in an additional layer, for example in the intermediate layer 13, extends from a perforation location, for example the inlet 3, to a circumferential end of said additional layer, for example the outlet 19.

In a third step of the manufacturing method, the base-conductors complex comprising the base layer obtained from step I, is assembled by colamination, with the additional layer, such that the electrodes are arranged in the cut region. For example, the intermediate layer 13 is laminated on the base-conductors complex of FIG. 2B comprising the base layer 11 and the conductors 27a, 27b.

In a fourth step of the manufacturing method, the microfluidic channel is closed with another additional layer by colamination. For example, the cover layer 15 closes the microfluidic channel cut and delimited in the intermediate layer 13 and assembled with the base-conductors complex comprising the base layer 11 and the electrical conductors 27a, 27b. In an example, the cover layer 15 is perforated and then selectively laminated on the intermediate layer 13, so as to delimit an access opening to the connecting portions 25a, 25b. Cutting and colamination can advantageously be carried out in line on a converting machine.

In an alternative embodiment, one of the additional layers can also correspond to a product coming from one of the methods described above, for example the product coming from step H or I. For example, the cover layer 15 can be a base-conductors complex comprising an electrical conductor 27a′. In this embodiment, the additional layer and the first base-conductors complex comprising the base layer 11 and a conductor 27b′ can be joined, thanks to another additional adhesive layer, for example the intermediate layer 13, cut to the pattern of the desired microfluidic channel. This embodiment can, for example, be implemented to obtain the configuration represented in FIGS. 4A and 4B. In another example, the cover layer 15 can be a base-conductors complex comprising electrical conductors 27a″ and 27b″. In this embodiment, not represented, the cover layer 15 and the base layer 11 can be joined, thanks to another additional adhesive layer, for example the intermediate layer 13, cut to the pattern of the desired microfluidic channel. In this embodiment, the connecting portions 25a″, 25b″ can be located on the external connecting surface 17 and the electrodes 23a″, 23b″ on the opposite face of the layer 15. Metallised holes or vias are thus used to electrically connect the connecting portions 25a″, 25b″ and the electrodes 23a″, 23b″.

The additional layers can be layers of materials identical to or different from one other and the material of the base layer. In particular, in a preferred embodiment, an additional layer can be a layer of adhesive material to hold the base layer together with another additional layer superimposed. For example, the material of the intermediate layer 13 is different from the material of the base 11 and cover 15 layers.

The method described makes it possible to economically and rapidly obtain a device having the features and advantages of the device 100. In particular, this method can be implemented on a large scale, for example in a reel-to-reel method, and it is possible to obtain sufficient cost efficiency to provide a consumable and replaceable device for capturing perspiration.

### NUMERICAL REFERENCES

1 contact surface of the device
3 inlet to microfluidic channel
5 microfluidic channel
7 groove in the contact surface
9 multilayer structure
11 base layer
13 intermediate layer
15 cover layer
17 external connecting surface
19 microfluidic channel outlet
21 corner of the device
22a, 22b surfaces adjacent to the contact surface
23a, 23b electrodes at the first ends of the conductors
25a, 25b connecting portions at the second ends of the conductors
27a, 27b electrical conductors
29 surface of the base layer opposite the contact surface
31 surface of the cover layer opposite the external connecting surface
100, 100′ devices for capturing perspiration
200 electronic device
300a, 300b parts of a wristwatch
1000 device for measuring perspiration
M3 through hole according to the manufacturing method
M11 base layer according to the manufacturing method
M12 copper layer according to the manufacturing method
M14 photosensitive resin layer according to the manufacturing method
M14a resin polymerisation zone according to the manufacturing method
M14b non-polymerised zone of the resin according to the manufacturing method
M16 light beam according to the manufacturing method
M18 base-conductors complex according to the manufacturing method
M27a, M27b electrical conductors according to the manufacturing method

1. A device configured to capture perspiration from a perspiring body and to be attached to an electronic device, comprising:
a multilayer structure having a contact surface configured to be placed in contact with the body,
the multilayer structure comprising an inner microfluidic channel extending through the multilayer structure and fluidly connecting at least one fluid inlet to at least one fluid outlet,
the inlet (3) being at said contact surface, and the outlet being located at another surface different from said contact surface, and
at least one pair of electrical conductors, each conductor comprising:
at a first end, an electrode having a surface area (S) extending along a wall of the microfluidic channel, and
at a second end, a connecting portion, the connecting portion being accessible by an adjacent surface or a surface opposite the contact surface (1) and being configured to be electrically coupled to the electronic device.

2. The device according to claim 1, wherein the microfluidic channel (5) extends in a straight line from the fluid inlet (3) to the fluid outlet (19) of the multilayer structure (9).

3. The device according to claim 1, wherein the contact surface comprises at least one groove for directing the perspiration to the fluid inlet.

4. The device according to claim 1, wherein the respective electrodes of the pair of conductors extend along one same wall of the microfluidic channel.

5. The device according to claim 1, wherein the respective electrodes of the pair of conductors extend along opposite walls of the microfluidic channel.

6. The device according to claim 1, wherein the structure has a parallelepiped shape, in particular a flat rectangular parallelepiped shape.

7. The device according to claim 1, wherein the ratio (L/S) between the distance (L) separating the electrodes and the surface area (S) of the electrodes is between 0.01 and 0.1 m\hat{ }-1, in particular between 0.03 and 0.05 m\hat{ }-1.

8. The device according to claim 1, wherein the multilayer structure comprises a base layer, an intermediate layer and a cover layer,
the base layer comprising the contact surface configured to be placed in contact with the body, and
the intermediate layer being disposed between the base layer and the cover layer.

9. The device according to claim 8, wherein the microfluidic channel is provided in the intermediate layer.

10. The device according to claim 1, characterized in that the multilayer structure comprises only three layers.

11. The device according to claim 1, characterized by the absence of electronic components.

12. A perspiration measurement appliance, comprising a device according to claim 1 and an electronic device,
the device being attached to the electronic device, the connecting portions of at least one pair of conductors of the device being electrically coupled to the electronic device, and the electronic device being configured to measure an electrical conductance and/or an electrical resistance between the electrodes of the device.

13. The method for manufacturing a device according to claim 1, comprising the steps of:
(a.) Providing a base layer and manufacturing at least one pair of electrical conductors on the base layer, each conductor comprising at a first end, an electrode, and at a second end, a connecting portion,
(b.) Providing an additional layer comprising at least one cut region forming a microfluidic channel, and
(c.) Assembling, in particular by colamination, the base layer and said at least one additional layer, such that the electrodes are arranged in the cut region.

14. The method according to claim 13, comprising an additional step of:
(d.) Closing the microfluidic channel with another additional layer, in particular by colamination.

15. The method according to claim 13, comprising an additional step of:
(e.) Producing a through hole in the base layer forming an inlet to the microfluidic channel.

16. The method according to claim 14, the cut region forming a microfluidic channel comprises an access point to the through hole forming an inlet.

17. The method according to claim 13, wherein step (a.) comprises the steps of:
i) Providing a base layer,
ii) Covering the base layer with a layer of copper,
iii) Applying a photosensitive resin layer at least on the copper layer,
iv) Selectively exposing the photosensitive resin to a light beam, in particular an ultraviolet beam, so as to delimit a path of the conductors,
v) Dissolving the non-exposed photosensitive resin, and
vi) Chemically etching the copper layer, so as to obtain a base-conductors complex.

18. The method according to claim 13, wherein step (a.) comprises the steps of:
i) Providing a base layer, and
ii) Printing the conductors on the base layer.

19. The method according to claim 13, comprising the additional step of:
x) Plating the conductors, in particular the electrodes, with at least one metal, in particular a metal selected from among nickel, gold, silver and palladium, or with a metal alloy, in particular a metal alloy comprising at least one metal selected from among nickel, gold, silver and palladium.

20. The method according to claim 19, wherein the plating of step x) is a plating with a nickel layer covered with a gold layer.
